(19) United States
(12) Patent Application Publication
(10) Pub. No.: US 2020/0279635 A1
(43) Pub. Date: Sep. 3, 2020

(54) SYSTEM FOR SUPPORTING CLINICAL DECISION-MAKING IN REPRODUCTIVE ENDOCRINOLOGY AND INFERTILITY

(71) Applicants: Gerard Letterie, Seattle, WA (US); Andrew MacDonald, Seattle, WA (US)

(72) Inventors: Gerard Letterie, Seattle, WA (US); Andrew MacDonald, Seattle, WA (US)

(21) Appl. No.: 16/650,333

(22) PCT Filed: Sep. 25, 2018
(86) PCT No.: PCT/US18/52611

§ 371 Date: Mar. 24, 2020

Related U.S. Application Data
(60) Provisional application No. 62/562,994, filed on Sep. 25, 2017.

(51) Int. Cl. G16H 20/40; G16H 10/60; G16H 10/40; G16H 20/10; G16H 50/20; G16H 50/50; G16H 15/00

(52) U.S. Cl. CPC G16H 20/40; G16H 10/60; G16H 10/40; G16H 15/00; G16H 50/20; G16H 50/50; G16H 20/10

(57) ABSTRACT

A computer system is configured to support clinical decision-making associated with patient treatment during the course of, e.g., an ovarian stimulation cycle. The system includes one or more computing devices programmed to receive patient training data; create decision model(s) using the patient training data; receive patient input data for at least one patient; provide the patient input data as input to the decision model(s); obtain output from the decision model(s); and generate recommendations for patient treatment for presentation via a user interface based on the output of the decision model. The decision model(s) may be created using random decision forests. The output from the decision model(s) may include confidence percentages for potential outcomes. The recommendations may be generated based on the confidence percentages.

### CROSS-REFERENCE(S) TO RELATED APPLICATION(S)

This application claims the benefit of U.S. Provisional Application No. 62/562,994, filed on Sep. 25, 2017, which is incorporated herein by reference.

### BACKGROUND

In general, clinical decision-making involves selecting appropriate actions to be taken for diagnosing and treating patients using clinical information and evidence-based decisions to arrive at a recommended course of action. In treating infertility, it is desirable that such decisions yield the highest likelihood of pregnancy and advance the well-being of the patient while remaining fully aware of and weighing the alternate approaches and risks associated with diagnostic and treatment processes.

A typical in vitro fertilization (IVF) process involves administering medications to mature as many eggs as possible in a safe manner. The ovarian response is measured with a sequential series of lab testing for the hormone estradiol (E2) and ultrasound examinations measuring ovarian follicles in two dimensions (e.g., in mm), reported as the mean in the context of the number of days of ovarian stimulation. These two observations measure ovarian response to the medications of recombinant FSH (follicle stimulating hormone) and LH (luteinizing hormone)/FSH combinations, among others. An early decision in this process is whether to stop the administration of medications or continue with medications to stimulate the ovary. If the decision is to stop, the cycle may be ended without further intervention (cycle cancellation) or with a trigger injection of hCG (human chorionic gonadotropin), lupron or ovidrel (cycle trigger) to mature the oocytes and prepare them for retrieval. The medication and dose used for the trigger may be predetermined and may not be relevant to making the decision to stop or continue stimulating the ovary.

Because selection of treatment stimulation protocol (e.g., a combination of drugs such as recombinant FSH, combination FSH/LH, leuprolide acetate, cetrotide and ganerelix) involves weighing risks, and because all information (such as underlying causes) may not be known, there is uncertainty involved in the process of making clinical decisions. Protocol selection and follow up are made qualitatively on a daily basis and based on a clinician's/provider's understanding of response and using clinical impressions based on experience. Consequently, attempts are often made to quantify and constrain the effect of making clinical decisions in an effort to reduce uncertainty.

Like many medical practices, clinical decision making in reproductive endocrinology and infertility treatments can be supported by electronic medical records (EMR), clinical databases, and the like. Although a number of systems exist in the prior art to perform data mining, trend extraction, and determine relationships in data, none have the effectiveness or benefits of the embodiments described herein.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In one aspect, a computer system is configured to support clinical decision-making associated with patient treatment during the course of an ovarian stimulation cycle. The system includes one or more computing devices programmed to receive patient training data for a plurality of patients; create at least one decision model (e.g., a regression model) using the patient training data; receive patient input data for at least one patient; provide the patient input data as input to the at least one decision model; obtain output from the decision model; and generate at least one recommendation for patient treatment for presentation via a user interface based on the output of the decision model. In an embodiment, the at least one decision model is created using a set of one or more random decision forests. Two or more decision models may be used, and each of the decision models may be created using a different random decision forest. The output from the at least one decision model may include one or more confidence percentages for one or more potential outcomes, and the at least one recommendation may be generated based on the one or more confidence percentages.

In an embodiment, four decision models are used for different aspects of clinical decisions, and the set of random decision forests includes four random decision forests. The decision models are for obtaining decisions on, respectively, (1) continuing or canceling an ovarian stimulation cycle, (2) if the cycle is to continue, whether to trigger egg retrieval or to continue stimulation, (3) if stimulation is to be continued, the date of the next monitoring appointment, and (4) if stimulation is to be continued, whether the dose of stimulation medication is to be increased or decreased, or remain the same. Data points for the decision may be associated with patient visits to a clinic. In an embodiment, the data points include one or more of the following inputs: age of the patient when the cycle began; body mass index of patient when the cycle began; protocol being followed for the cycle; patient diagnosis category; visit number in the cycle; number of days elapsed since the beginning of the cycle; measured estradiol levels (e.g., from a current visit or one or more previous visits); previously administered stimulation doses; measured diameters of one or more developing follicles.

In another aspect, a computer system implements a method that uses a repository of patient medical records in supporting clinical decision-making during in vitro fertilization (IVF). The method includes receiving patient data from a patient electronic medical record (EMR) database associated with treatment of infertility wherein the patient data comprises age, diagnosis, lab testing data prior to start of an IVF cycle, and observations during a process of ovarian stimulation; identifying, in the EMR database, information concerning different treatments previously employed for treating infertility; and based on the identified information, generating an order for patient treatment, wherein the order is associated with protocol selection, dose of medication, date of follow up, or cycle stop. In an embodiment, generating the order for patient treatment comprises providing the patient data as input to at least one decision model, wherein the at least one decision model is created using a set of one or more random decision forests. The information concerning different treatments may include one or more of diagnosis, laboratory data (e.g., FSH (follicle stimulating hormone), AMH (anti-mullerian hormone), estradiol and antral follicle count from baseline testing), maternal age, sperm parameters, and prior different treatments. The information concerning different treatments may include resource consumption characteristics, which may be selected from a group consisting of cost or duration of a treatment or course of treatment, physician or staff hours, cost or quantity of medicine, cost or duration of equipment usage, cost or duration of inpatient stay, or cost or duration of home care.

The order may be transmitted to a patient computing device or a clinician computing device. The transmission may be performed immediately or held pending approval by a physician. Orders or other medical information may be securely transmitted to a patient's computing device (e.g., a smartphone) via one or more modes of communication (e.g., SMS message, email, etc.), and such transmissions may be performed in compliance with applicable data protection or patient privacy laws.

The order may include one or more of (a) an order for a medical test to be made for a patient, (b) an order for a pharmacological prescription for a patient, (c) an order for a service to be performed for a patient SER (sonographic egg retrieval), (d) an order for diagnostic imaging to be performed for a patient, (e) an order for surgical treatment for a patient, or (f) an order for a form of physiological therapy to be performed on a patient.

The different treatment information may be analyzed to determine a difference in diagnosis or treatment associated with said order and corresponding previous diagnoses and treatments recorded for other patients. The system may be configured to generate a report of such analysis for display in a user interface.

In another aspect, a computer system is configured to use a repository of patient medical records in supporting clinical decision-making. The system includes one or more computing devices programmed to receive, from a source computing device, data representing an order associated with treatment of a medical condition; interpret the order to determine search criteria for use in identifying records related to the medical condition; initiate a search of a database of patient medical records based on the search criteria to identify treatment information concerning different treatments previously employed for treating the medical condition; and transmit the treatment information to the source computing device. The computer system may include the database of patient medical records, or the database may be provided by some other system. At least one of the one or more computing devices may be programmed to provide a user interface for use in supporting clinical decision making, the user interface being configured to receive user input and generate data representing the order associated with treatment of the medical condition.

In any of the described embodiments, computer-executable instructions may be stored on a computer-readable storage medium, and these instructions may be configured to cause one or more computing devices to perform any of the methods described herein. In any of the described embodiments, a computing device or a system comprising one or more computing devices may be programmed to perform any of the methods described herein.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is a flow chart that depicts an illustrative process of generating recommendations for patient treatment according to an embodiment of the present disclosure;

FIG. 2 is a flow chart that depicts an illustrative process of generating an order for patient treatment of infertility according to an embodiment of the present disclosure;

FIG. 3 is a flow chart that depicts an illustrative process of searching a database for patient treatment information according to an embodiment of the present disclosure;

FIG. 4 is a system diagram that depicts an illustrative computer system in which disclosed embodiments may be implemented; and

FIG. 5 is a block diagram that illustrates aspects of an illustrative computing device appropriate for use in accordance with embodiments of the present disclosure.

### DETAILED DESCRIPTION

In the following description, numerous specific details are set forth in order to provide a thorough understanding of illustrative embodiments of the present disclosure. It will be apparent to one skilled in the art, however, that many embodiments of the present disclosure may be practiced without some or all of the specific details. In some instances, well-known process steps have not been described in detail in order not to unnecessarily obscure various aspects of the present disclosure. Further, it will be appreciated that embodiments of the present disclosure may employ any combination of features described herein. The illustrative examples provided herein are not intended to be exhaustive or to limit the claimed subject matter to the precise forms disclosed.

Intelligent computing systems for supporting clinical decision-making in reproductive endocrinology and infertility are described. The systems can be used in the context of, e.g., day-to-day monitoring and scheduling of clinical care and dose of medication, determining method of use of the drugs and protocol selection, and determining percent success for any given cycle day of stimulation.

In embodiments described herein, a computer system uses a repository of patient medical records in supporting clinical decision making, and which incorporates data associated with treatment of infertility. In at least one embodiment, the system interprets clinical, ultrasound, and laboratory data to determine management of a cycle of in vitro fertilization. Such management may include decisions regarding starting protocol, scheduling appointments, and determining whether to cancel or continue ovarian stimulation.

In embodiments described herein, a database includes data contained in an electronic medical record (EMR) that may include, among other data points, the patient's demographics, past medical history and infertility evaluation including diagnosis, lab testing for ovarian reserve, and any radiologic studies pertinent to a diagnosis of infertility. The database of patient medical records can be queried to identify, e.g., information concerning prior diagnosis and lab studies/results, and treatments previously employed for infertility.

In general, clinical decision-making involves selecting appropriate actions to be taken for diagnosing and treating patients using clinical information and evidence based decisions to arrive at a recommended course of action. In treating infertility, it is desirable that such decisions yield the highest likelihood of pregnancy and advance the well-being of the patient while remaining fully aware of and weighing the alternate approaches and risks associated with diagnostic and treatment processes.

A typical IVF process involves administering medications to mature as many eggs as possible in a safe manner. The ovarian response is measured with a sequential series of lab testing for the hormone estradiol (E2) and ultrasound examinations measuring ovarian follicles in two dimensions (e.g., in mm), reported as the mean in the context of the number of days of ovarian stimulation. These two observations measure ovarian response to the medications of recombinant FSH and LH/FSH combinations, among others. An early decision in this process is whether to stop the administration of medications or continue with medications to stimulate the ovary. If the decision is to stop, the cycle may be ended without further intervention (cycle cancellation) or with a trigger injection of hCG, lupron or ovidrel (cycle trigger) to mature the oocytes and prepare them for retrieval. The medication and dose used for the trigger may be predetermined and may not be relevant to making the decision to stop or continue stimulating the ovary.

Described computer systems may be used for, e.g., day-to-day monitoring and scheduling of clinical care and dose of medication, determining method of use of the drugs and protocol selection, and determining likelihood of success (expressed as retrieving oocytes, developing embryos and pregnancy), e.g., for a given response for a specific day of ovarian stimulation.

Because selection of treatment stimulation protocol (e.g., a combination of drugs such as recombinant FSH, combination FSH/LH, leuprolide acetate, cetrotide and ganerelix) involves weighing risks, and because all information (such as underlying causes) may not be known, there is uncertainty involved in the process of making clinical decisions. Protocol selection and follow up are made qualitatively on a daily basis and based on a clinician's/provider's understanding of response and using clinical impressions based on experience. Consequently, attempts are often made to quantify and constrain the effect of making clinical decisions in an effort to reduce uncertainty.

In described embodiments, by accessing large databases and archival data, protocols may be identified that offer the best and safest outcomes. Data regarding treatment protocol selection and monitoring is extracted from an archival database and can be used, for example, to predict desirable outcomes in protocol selection and monitoring and to make accurate and cost-effective decisions regarding degree and intensity of monitoring (e.g., frequency and scheduling regarding when monitoring return visits are needed or can be most effectively ended). This has the effect of providing a quantitative understanding of the likelihood of success or failure, as well as the consequences associated with making various clinical decisions. Although a number of systems exist in the prior art to perform data mining, trend extraction, and determine relationships in data, none have the effectiveness or benefits of the embodiments described herein.

Medications are used in a combination of three of four in a stimulation protocol to stimulate the ovarian follicles to yield mature eggs (Group I) and one medication is used to induce final maturation of the oocytes in preparation for retrieval from the ovary and fertilization (Group II). Group I drugs include recombinant FSH, LH and FSH, leuprolide acetate, and cetrotide. Group II drugs include hCG. These drugs are administered over an 8 to 14-day interval (known as monitoring). Doses, start and end times, and follow up clinical examinations can be varied based on response. Clinical information can be compared with archival data to determine which protocol offers desirable outcomes and/or cost effectiveness. In at least one embodiment, the protocol is selected from among MDL (microdose Lupron), LL (long Lupron), antagonist, and luteal estrogen/antagonist protocols.

In described embodiments, four decisions are made to manage the clinical day to day monitoring of ovarian stimulation to develop the optimal number and quality of oocytes: (1) stop medication and the process of observation and end the stimulation (called cancellation), (2) stop the medications and administer hCG (trigger) to induce final maturation of the oocytes and plan oocyte retrieval in 36 hours, (3) continue to process and plan next steps regarding medication dose (e.g., increase, decrease, no change) and, (4) date for follow up appointment.

In described embodiments, a system will support major therapeutic decisions for a patient undergoing IVF, based on a larger population of patients previously treated in the patient's age group, such as: (1) treatment protocol and starting doses of medication, (2) the likelihood that the patient will need to be evaluated in a given time period (e.g., one, two, or three days), (3) dose adjustment, (4) whether to cancel and execute the binary decision of trigger or stop, and (5) the likelihood of success given a response for a given day of the stimulation cycle.

In described embodiments, a system receives, from a source computing device (e.g., a clinician's computing device), data representing an order associated with treatment of a medical condition; interprets the order to determine search criteria for use in identifying records related to the patient medical condition; searches a database of patient medical records based on the search criteria; identifies, in the patient medical record database, information concerning different treatments previously employed for treating the medical condition based on the search criteria; and provides the identified treatment information to the source computing device. The clinician's computing device may provide a graphical user interface that allows the user to, e.g., provide input relating to an order, and the device may generate the data representing the order in response to the user input. The clinician's computing device also may provide a user interface for viewing or analyzing patient records or decision recommendations, editing or transmitting related communications to patients, or other functionality.

Described embodiments incorporate patient data and clinical response during ovarian stimulation, accompanying methodology, and embedded analytical functions to provide physicians with feedback and clinical decisions regarding the next steps to achieve desirable outcomes in terms of number of high-quality and mature oocytes and clinical pregnancy rate.

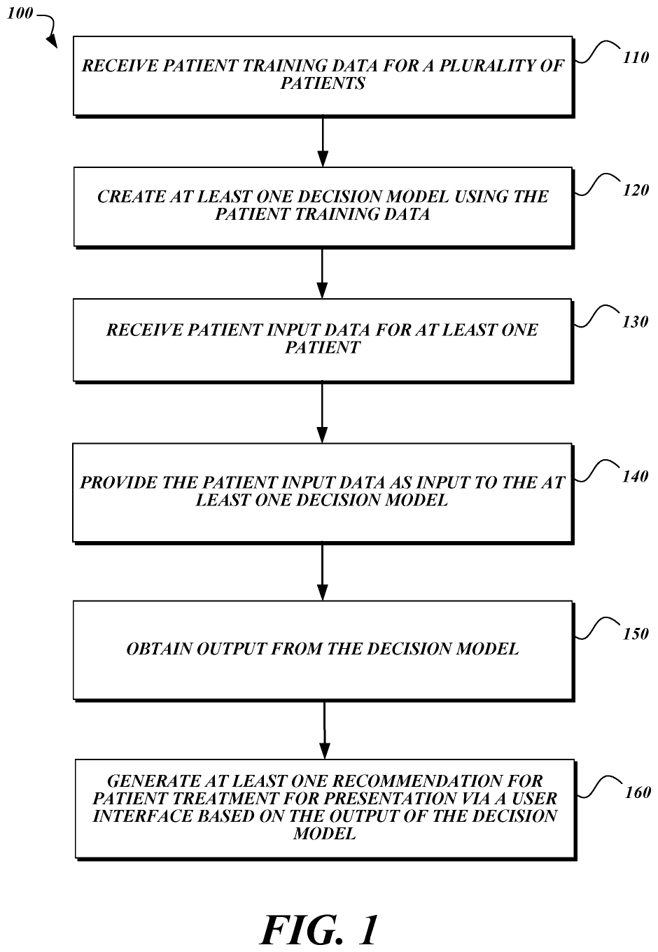

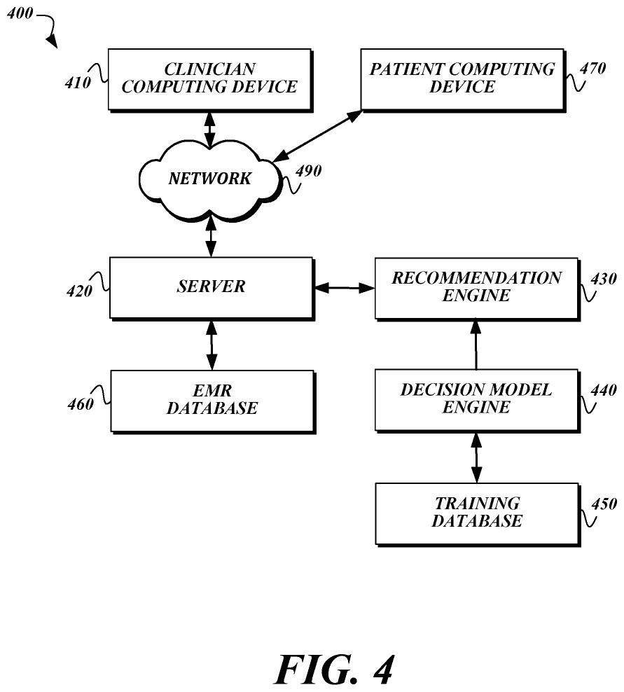

FIG. 1 is a flow chart that depicts an illustrative process 100 of generating recommendations for patient treatment according to an embodiment of the present disclosure. The process 100 may be implemented by the computer system 400 described below with reference to FIG. 4, or by some other system. At step 110, the system receives patient training data for a plurality of patients. At step 120, the system creates one or more decision models using the patient training data, as described in further detail below. The decision models, once trained, allow the system to generate recommendations for, e.g., treatment of medical conditions. Accordingly, at step 130 the system receives patient input data for a patient and at step 140, the system provides the patient input data as input to the decision models, as described in further detail below. At step 150, the system obtains output from the decision model, and at step 160 the system generates at least one recommendation for patient treatment for presentation via a user interface based on the output of the decision model. In an embodiment, the at least one decision model is created using a set of one or more random decision forests. Two or more decision models may be used, and each of the decision models may be created using a different random decision forest. The output from the at least one decision model may include one or more confidence percentages for one or more potential outcomes, and the at least one recommendation may be generated based on the one or more confidence percentages.

In one illustrative implementation, an expert system is trained with a database of patient data, and can make recommendations for patient treatment during the course of an ovarian stimulation cycle. The expert system is built from a set of decision forests trained to maximize predictive power for decisions such as (1) continuing or canceling the cycle, (2) if the cycle is to continue, whether the next step is to trigger egg retrieval or to continue stimulation, (3) if stimulation is to be continued, the date of the next monitoring appointment (e.g., 1, 2, or 3 days), and (4) if stimulation is to be continued, whether the dose of stimulation medication is to be increased, decreased, or remain the same.

Decision forests are constructed from multiple decision trees. The use of multiple decision trees in a decision forest provides an ensemble approach to learning that helps to avoid overfitting data in a single decision tree, which can provide misleading results. Thus, in described embodiments, decision forests can provide improvements over previous machine learning approaches in terms of predictive accuracy, accuracy of confidence, generalization, and computational efficiency. The decision forests may be implemented as random decision forests, in which individual decision trees in the forest are created with one or more randomization aspects (e.g., randomized feature selection, random sampling of training data, randomized node optimization) that makes the trees in the forest distinct from one another. A decision forest can be described in terms of parameters such as forest size (number of trees), tree depth, amount of randomness (correlation between trees in the forest), type of randomness, choice of learning models, training objectives, and choice of features or predictor variables. The decision of the decision forest can adjust such parameters to provide a desired level of accuracy, efficiency, and fit for the problem to be solved.

In described embodiments, the decision forests are trained with data gathered from prior stimulation cycles. Data points may be associated with a patient visit to a clinic and may include any combination of the following inputs:
1. Whether the cycle continued after the visit.
2. If the cycle continued, whether the next step was a trigger or continued stimulation.
3. If stimulation continued, how many days later was the next monitoring visit.
4. If stimulation continued, was the stimulation dose increased, decreased or held constant.
5. Age of the patient when cycle began.
6. Body mass index (BMI) of patient when cycle began (e.g., a particular BMI value, or an indication of whether a BMI value exceeds a specified threshold, such as 30).
7. Protocol followed for this cycle.
8. Patient diagnosis category (e.g., a category selected from a set of possible diagnosis categories, such as the following: anovulation, unexplained, tubal factor, endometriosis, male factor, diminished ovarian reserve, uterine factor, repeated pregnancy loss, PCOS (polycystic ovary syndrome), and “other”).
9. Visit number in present cycle.
10. Number of days elapsed since the beginning of the cycle.
11. Measured estradiol level from the current visit.
12. Measured estradiol levels from the previous two visits (if any).
13. Previously administered stimulation doses from the previous two visits (if any).
14. Measured diameters of the four largest developing follicles during the current visit (or fewer if four are not present).

During training, input data may be separated into two datasets. The first may be used to train the system, while the second may be held out of the training set and used to validate the generated model. Using the data, four separate models were trained and evaluated, one for each of the decision outcomes mentioned above.

In some embodiments, decision forests are used to generate decision models in the form of regression models. This approach allows the system to output continuous variables, such as confidence percentages, as explained in further detail below.

In an illustrative embodiment, each data point consists of the 14 input items listed above, with one decision forest for each of four decision models, although other input items or combinations of input items also may be used. In the illustrative embodiment, in each model inputs 5-14 were used as the training variables, and one of input 1-4 was used as the decision variable, depending on which decision was being trained. Thus, the four models are: (1) decision to continue treatment, (2) decision to trigger egg retrieval or continue stimulation, (3) decision of date of next monitoring appointment, (4) decision of increasing, decreasing, or holding constant the medication dose. In an embodiment, random decision forests are used for each decision model, with design of each forest being adapted to the particular decision to be made. For example, in decision 1 (deciding whether to continue treatment) a random decision forest may be used in which the number of trees is 2,000 and the number of predictor variables is 10. For decisions 2 and 4 (whether to trigger egg retrieval and setting date for the next appointment) a random decision forest may be used in which the number of trees is 500 and the number of predictor variables is 4. For decision 3 (medication dose), a random decision forest may be used in which the number of trees is 1,000 and the number of predictor variables is 17. These forest parameters are only illustrative; these parameters may be adjusted in other ways, or other parameters may be adjusted to tune the random decision forests for each decision model.

Each model provides a recommendation as a confidence percentage for each potential outcome; the percentages may be used directly in some applications, such as by providing a confidence percentage for a particular outcome (or a set of possible outcomes) directly to a clinician for review; in other applications, the highest-percentage outcome for a given model may be employed as the recommendation for that model, in which a recommended action may be provided without the corresponding confidence percentage.

In an illustrative embodiment, the model can be employed by providing data collected during a patient monitoring visit, corresponding to inputs 5-14 above. These inputs are fed to each of the four decision models and a prediction is made for each decision. These predictions are used to inform the care of the patient when deciding the next steps to take in the cycle. If model 1 indicates treatment should continue (e.g., because the “continue” confidence percentage is greater than the “discontinue” confidence percentage), then the result of model 2 is employed to decide on the next course of action. If model 2 recommends further stimulation, the results of models 3 and 4 are used to recommend how that stimulation should proceed. If model 2 recommends trigger, the results of models 3 and 4 are not needed. If model 1 recommends discontinuing the cycle, output from the other three models is not needed.

In some cases, all of inputs 5-14 may not be available, or if all are available, it may not be necessary for all of the inputs to be used. For example, if input 6 (BMI of patient when cycle began) is not known, such as where the body mass index was not measured when the cycle began, this input may be estimated based on a current body mass index, or omitted from the data provided. Further, it may be the case that as the system is used over time, additional inputs may be included or inputs that appear not to be relevant to the particular clinical decision may be omitted. The decision models can be adjusted or retrained as inputs are added or removed from the data set.

In addition, some inputs may be more important than others for particular decisions. The number and type of inputs to be used may, therefore, depend to some extent on the decision to be made by the system. For example, in a system where dosage level is not decided by the system, the BMI input may have less importance if BMI is not predictive in the other decisions to be made. On the other hand, the cycle day of the particular visit may be important for all four decision models and may be included as an input for each model.

In an embodiment, the day of the visit (cycle day) was found to be a highly significant variable for decision 1 (whether to continue treatment), with visit number, estradiol measurements (current and previous), and follicle diameters also having significance. For decision 2 (trigger decision) estradiol measurements, follicle diameters, and the day of the visit were found to be significant. For decision 3 (medication dose) estradiol measurements, BMI, follicle diameter, day of visit, and age of the patient were found to be significant. For decision 4 (setting date for next appointment), follicle diameter was found to be significant, as were day of visit and estradiol measurements.

Described embodiments may be designed to employ supervised learning or some combination of supervised and unsupervised learning. In supervised learning, the system maps inputs to outputs based on known inputs and outputs. Thus, supervised learning can be used where previous inputs (e.g., data points including inputs 5-14 described above) and corresponding clinical outcomes are known. In some embodiments, decision forests are used, as described above. Other illustrative algorithms and approaches that may be used in supervised learning include decision trees, neural networks, regression models, and vector machines. In unsupervised learning, the system infers functions from unclassified or uncategorized data. Thus, unsupervised learning may be useful for analysis of input data in which clinical outcomes are not known. Illustrative algorithms that may be used in unsupervised learning include clustering algorithms, anomaly detection algorithms, and neural networks.

Described embodiments may be specialized, in which the system is trained using clinic-specific data, or generalized with a fully loaded and trained system deployed to a practice for immediate use. Software for the system may be made available for download in a cloud computing environment. In this regard, described systems may require no additional hardware beyond what may be used for other computing tasks in a clinic, such as accessing electronic medical records.

Described embodiments may operate in combination with other systems (e.g., secure servers hosting electronic medical records systems) over a communication network, such as, for example, the Internet or an intranet, or may operate as a stand-alone system. Security measures, such as encryption, may be employed to preserve patient privacy. Described embodiments may be deployed, at least in part, to end users as client applications on client computing devices (e.g., in communication with a server), and may be deployed over a closed network, virtual private network, or any other networked system. Described embodiments may be implemented as a “plug in” or stand-alone application.

In an illustrative embodiment, an automated control workflow is used to compare results obtained from data computed in the reference model (i.e., an expected behavior model based on historical record) with test data for the particular patient under examination. Results of the diagnostic testing used for input to the model (e.g., inputs 5-14) and the decisions regarding next steps are provided as output for analysis and are captured within the patient's record. The physician can examine the decision recommendations and transmit them to the patient as calculated or make adjustments based on experience or other factors before transmitting.

In addition, historical data from archives, the patient's personal file, and experience (e.g., prior IVF cycles) associated with other similar patients within the physician's care or other patients undergoing IVF may be compared with the results of the patient under review. An indication can be provided as to whether significant deviations exist between this patient and the larger population regarding the observations for specific cycle days. In this way, the physician has access to both previous records of his or her patients and to a larger population with which to evaluate and scrutinize the quality of patient care, and to affirm present orders in light of past history.

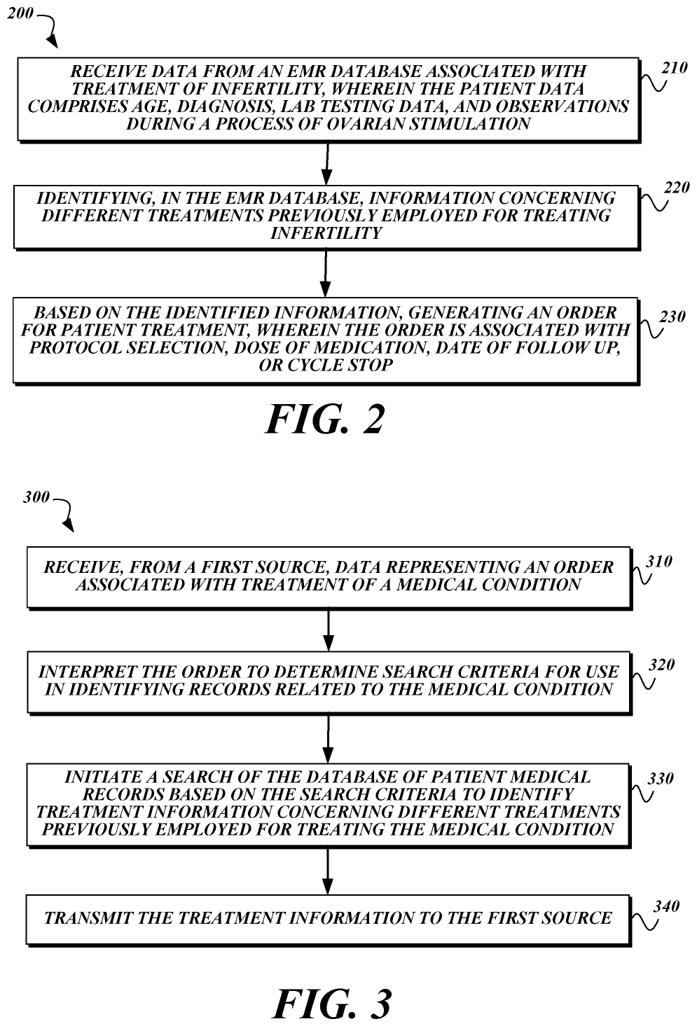

FIG. 2 is a flow chart that depicts an illustrative process 200 of generating an order for patient treatment of infertility according to an embodiment of the present disclosure. The process 200 may be implemented in the computer system 400 described below with reference to FIG. 4, or in some other system. In this example, the system implements a method that uses a repository of patient medical records in supporting clinical decision-making during in vitro fertilization (IVF). At step 210, the system receives patient data from a patient electronic medical record (EMR) database associated with treatment of infertility wherein the patient data comprises age, diagnosis data, lab testing data prior to start of an IVF cycle, and observations during a process of ovarian stimulation. At step 220, the system identifies, in the EMR database, information concerning different treatments previously employed for treating infertility. At step 230, the system generates an order for patient treatment based on the identified information. The order is associated with protocol selection, dose of medication, date of follow up, or cycle stop. In an embodiment, generating the order for patient treatment comprises providing the patient data as input to at least one decision model, wherein the at least one decision model is created using a set of one or more random decision forests. The information concerning different treatments may include one or more of diagnosis, laboratory data (e.g., FSH (follicle stimulating hormone), AMH (anti-mullerian hormone), estradiol and antral follicle count from baseline testing), maternal age, sperm parameters, and prior different treatments. The information concerning different treatments also may include resource consumption characteristics, as described in detail below.

FIG. 3 is a flow chart that depicts an illustrative process 300 of searching a database for patient treatment information according to an embodiment of the present disclosure. The process 300 may be implemented in the computer system 400 described below with reference to FIG. 4, or in some other system. In the illustrative workflow described in FIG. 3, the system receives data representing an order associated with treatment of a medical condition from a first source (e.g., a clinician's computing device) at step 310. At step 320, the system interprets the order to determine search criteria for use in identifying records related to the patient's medical condition. This may involve parsing the order for keywords and patient identifying information and including such information and keywords in a search request. To assist in this process, the order may be provided in a structured form. The structure may be provided, for example, by a web form in which the information may be entered in designated fields by the clinician. At step 330, the system initiates a search of a database or repository of patient medical records based on the search criteria, and the system identifies information concerning different treatments previously employed for treating the medical condition based on the search criteria in the patient medical record database. At step 340, the system transmits the treatment information to the first source. The treatment information may be formatted and displayed via a computing device, such as a tablet or notebook computer, smart phone, or other computing device.

The information concerning different treatments may include resource consumption characteristic information concerning the different treatments, so that the resource consumption characteristics concerning treatment for a particular patient may be compared with corresponding resource consumption characteristics for the different treatments. These resource consumption characteristics may include, for example, financial cost of a treatment or course of treatment, quantity of medicine, number of nursing hours, number of physician hours, cost of equipment usage, length of time equipment is used, length of time of inpatient stay, duration of home care facility usage and cost of medicine. Aggregating predictions of treatment across all active patients in a practice can result in cost reduction through more accurate prediction of future resource needs, both materials and labor, so unneeded resources are not purchased. This use may be enhanced by use of an Enterprise Resource Planning (ERP) system or other inventory management system.

In an additional embodiment, the confidence percentage of model 1 (described above) can be used to predict likelihood of overall success of the cycle at each visit by comparing to the model 1 confidences of previous cycles with successful and unsuccessful outcomes. The patient and physician can employ this likelihood prediction to decide to terminate a cycle early in order to preserve resources for use in subsequence cycles.

In an additional embodiment, a physician may run the model for a patient several times, varying input 7 (stimulation protocol) and may use the confidence percentages returned to determine which protocol is likeliest to achieve success for the patient.

Those of ordinary skill in the art will appreciate that the invention also includes the option for physician review of medication orders that the system assigns to ascertain the appropriateness of new orders or of similar treatments for specific classes of patients, and quality control review of patients to ascertain whether medications, timing of follow up and amount of medication that the system has assigned for a specific patient is consistent with approaches normally taken by physicians within the international fertility community.

FIG. 4 is a system diagram that depicts an illustrative computer system 400 in which disclosed embodiments, such as the processes depicted in FIGS. 1-3, may be implemented. In the example shown in FIG. 4, a clinician's computing device 410 communicates with a server computer 420 via a network 490. The server computer 420 can be viewed as a gateway between the clinician's computing device 410 and functionality provided by a recommendation engine 430, a decision model engine 440, a training database 450, and an EMR database 460, which may themselves be hosted by one or more other computers (e.g., in a cloud computing arrangement). The recommendation engine 430, the decision model engine 440, and the training database 450 may provide functionality described with reference to FIG. 1. In this example, the decision model engine 440 creates decision models using data provided by the training database 450 using techniques described above. The recommendation engine 430 uses these decision models to generate recommendations for treatment using techniques described above. The recommendation engine 430 receives patient data from the clinician computing device 410 and/or the optional patient computing device 470 via the server 420. The recommendation engine 430 provides the patient data as input to the decision models and obtains corresponding output from those models, e.g., in the form of confidence percentages as described above. The recommendation engine 430 then generates at least one recommendation for patient treatment, which can be transmitted to the clinician computing device 410 or the patient computing device 470 via the server 420.

The system 400 also may perform the order generation functionality described with reference to FIG. 2, or the database searching functionality described with reference to FIG. 3. The order generation and database searching functionality may be provided, for example, by an application running on the clinician computing device 410 or by an application running on the server 420 (which may, in turn, be accessed by a browser running on the clinician computing device 410). In either case, the server 420 may communicate with the EMR database 460 to obtain the desired information.

### Illustrative Devices and Operating Environments

Unless otherwise specified in the context of specific examples, described techniques and tools may be implemented by any suitable computing device or set of devices.

In any of the described examples, an engine may be used to perform actions. An engine includes logic (e.g., in the form of computer program code) configured to cause one or more computing devices to perform actions described herein as being associated with the engine. For example, a computing device can be specifically programmed to perform the actions by having installed therein a tangible computer-readable medium having computer-executable instructions stored thereon that, when executed by one or more processors of the computing device, cause the computing device to perform the actions. The particular engines described herein are included for ease of discussion, but many alternatives are possible. For example, actions described herein as associated with two or more engines on multiple devices may be performed by a single engine. As another example, actions described herein as associated with a single engine may be performed by two or more engines on the same device or on multiple devices.

In any of the described examples, a data store contains data as described herein and may be hosted, for example, by a database management system (DBMS) to allow a high level of data throughput between the data store and other components of a described system. The DBMS may also allow the data store to be reliably backed up and to maintain a high level of availability. For example, a data store may be accessed by other system components via a network, such as a private network in the vicinity of the system, a secured transmission channel over the public Internet, a combination of private and public networks, and the like. Instead of or in addition to a DBMS, a data store may include structured data stored as files in a traditional file system. Data stores may reside on computing devices that are part of or separate from components of systems described herein. Separate data stores may be combined into a single data store, or a single data store may be split into two or more separate data stores.

Some of the functionality described herein may be implemented in the context of a client-server relationship. In this context, server devices may include suitable computing devices configured to provide information and/or services described herein. Server devices may include any suitable computing devices, such as dedicated server devices. Server functionality provided by server devices may, in some cases, be provided by software (e.g., virtualized computing instances or application objects) executing on a computing device that is not a dedicated server device. The term “client” can be used to refer to a computing device that obtains information and/or accesses services provided by a server over a communication link. However, the designation of a particular device as a client device does not necessarily require the presence of a server. At various times, a single device may act as a server, a client, or both a server and a client, depending on context and configuration. Actual physical locations of clients and servers are not necessarily important, but the locations can be described as “local” for a client and “remote” for a server to illustrate a common usage scenario in which a client is receiving information provided by a server at a remote location. Alternatively, a peer-to-peer arrangement, or other models, can be used.

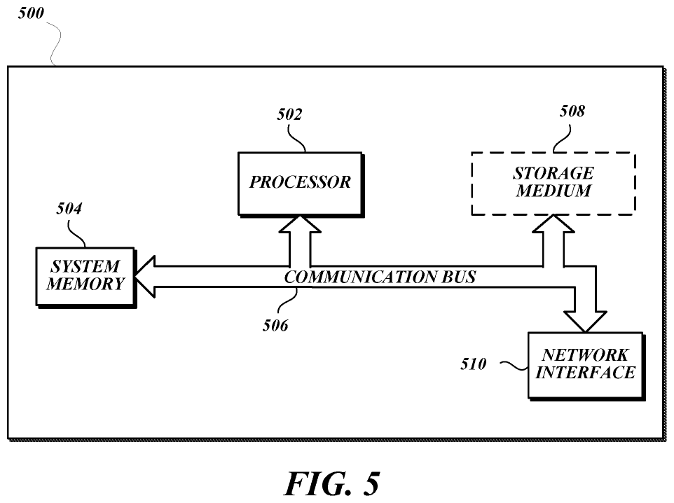

FIG. 5 is a block diagram that illustrates aspects of an illustrative computing device 500 appropriate for use in accordance with embodiments of the present disclosure. The description below is applicable to servers, personal computers, mobile phones, smart phones, tablet computers, embedded computing devices, and other currently available or yet-to-be-developed devices that may be used in accordance with embodiments of the present disclosure.

In its most basic configuration, the computing device 500 includes at least one processor 502 and a system memory 504 connected by a communication bus 506. Depending on the exact configuration and type of device, the system memory 504 may be volatile or nonvolatile memory, such as read only memory (“ROM”), random access memory (“RAM”), EEPROM, flash memory, or other memory technology. Those of ordinary skill in the art and others will recognize that system memory 504 typically stores data and/or program modules that are immediately accessible to and/or currently being operated on by the processor 502. In this regard, the processor 502 may serve as a computational center of the computing device 500 by supporting the execution of instructions.

As further illustrated in FIG. 5, the computing device 500 may include a network interface 510 comprising one or more components for communicating with other devices over a network. Embodiments of the present disclosure may access basic services that utilize the network interface 510 to perform communications using common network protocols. The network interface 510 may also include a wireless network interface configured to communicate via one or more wireless communication protocols, such as WiFi, 2G, 3G, 4G, LTE, WiMAX, Bluetooth, and/or the like.

In the illustrative embodiment depicted in FIG. 5, the computing device 500 also includes a storage medium 508. However, services may be accessed using a computing device that does not include means for persisting data to a local storage medium. Therefore, the storage medium 508 depicted in FIG. 5 is optional. In any event, the storage medium 508 may be volatile or nonvolatile, removable or nonremovable, implemented using any technology capable of storing information such as, but not limited to, a hard drive, solid state drive, CD-ROM, DVD, or other disk storage, magnetic tape, magnetic disk storage, and/or the like.

As used herein, the term “computer-readable medium” includes volatile and nonvolatile and removable and nonremovable media implemented in any method or technology capable of storing information, such as computer-readable instructions, data structures, program modules, or other data. In this regard, the system memory 504 and storage medium 508 depicted in FIG. 5 are examples of computer-readable media.

For ease of illustration and because it is not important for an understanding of the claimed subject matter, FIG. 5 does not show some of the typical components of many computing devices. In this regard, the computing device 500 may include input devices, such as a keyboard, keypad, mouse, trackball, microphone, video camera, touchpad, touchscreen, electronic pen, stylus, and/or the like. Such input devices may be coupled to the computing device 500 by wired or wireless connections including RF, infrared, serial, parallel, Bluetooth, USB, or other suitable connection protocols using wireless or physical connections.

In any of the described examples, input data can be captured by input devices and processed, transmitted, or stored (e.g., for future processing). The processing may include encoding data streams, which can be subsequently decoded for presentation by output devices. Media data can be captured by multimedia input devices and stored by saving media data streams as files on a computer-readable storage medium (e.g., in memory or persistent storage on a client device, server, administrator device, or some other device). Input devices can be separate from and communicatively coupled to computing device 500 (e.g., a client device), or can be integral components of the computing device 500. In some embodiments, multiple input devices may be combined into a single, multifunction input device (e.g., a video camera with an integrated microphone). The computing device 500 may also include output devices such as a display, speakers, printer, etc. The output devices may include video output devices such as a display or touchscreen. The output devices also may include audio output devices such as external speakers or earphones. The output devices can be separate from and communicatively coupled to the computing device 500, or can be integral components of the computing device 500. Input functionality and output functionality may be integrated into the same input/output device (e.g., a touchscreen). Any suitable input device, output device, or combined input/output device either currently known or developed in the future may be used with described systems.

In general, functionality of computing devices described herein may be implemented in computing logic embodied in hardware or software instructions, which can be written in a programming language, such as C, C++, COBOL, JAVA™ PHP, Perl, Python, Ruby, HTML, CSS, JavaScript, VBScript, ASPX, Microsoft.NET™ languages such as C#, and/or the like. Computing logic may be compiled into executable programs or written in interpreted programming languages. Generally, functionality described herein can be implemented as logic modules that can be duplicated to provide greater processing capability, merged with other modules, or divided into sub-modules. The computing logic can be stored in any type of computer-readable medium (e.g., a non-transitory medium such as a memory or storage medium) or computer storage device and be stored on and executed by one or more general-purpose or special-purpose processors, thus creating a special-purpose computing device configured to provide functionality described herein.

### Extensions and Alternatives

Many alternatives to the systems and devices described herein are possible. For example, individual modules or subsystems can be separated into additional modules or subsystems or combined into fewer modules or subsystems. As another example, modules or subsystems can be omitted or supplemented with other modules or subsystems. As another example, functions that are indicated as being performed by a particular device, module, or subsystem may instead be performed by one or more other devices, modules, or subsystems. Although some examples in the present disclosure include descriptions of devices comprising specific hardware components in specific arrangements, techniques and tools described herein can be modified to accommodate different hardware components, combinations, or arrangements. Further, although some examples in the present disclosure include descriptions of specific usage scenarios, techniques and tools described herein can be modified to accommodate different usage scenarios. Functionality that is described as being implemented in software can instead be implemented in hardware, or vice versa.

Many alternatives to the techniques described herein are possible. For example, processing stages in the various techniques can be separated into additional stages or combined into fewer stages. As another example, processing stages in the various techniques can be omitted or supplemented with other techniques or processing stages. As another example, processing stages that are described as occurring in a particular order can instead occur in a different order. As another example, processing stages that are described as being performed in a series of steps may instead be handled in a parallel fashion, with multiple modules or software processes concurrently handling one or more of the illustrated processing stages. As another example, processing stages that are indicated as being performed by a particular device or module may instead be performed by one or more other devices or modules.

The principles, representative embodiments, and modes of operation of the present disclosure have been described in the foregoing description. However, aspects of the present disclosure which are intended to be protected are not to be construed as limited to the particular embodiments disclosed. Further, the embodiments described herein are to be regarded as illustrative rather than restrictive. It will be appreciated that variations and changes may be made by others, and equivalents employed, without departing from the spirit of the present disclosure. Accordingly, it is expressly intended that all such variations, changes, and equivalents fall within the spirit and scope of the claimed subject matter.

1. A computer system configured to support clinical decision-making associated with patient treatment during the course of an ovarian stimulation cycle, the system comprising:
one or more computing devices programmed to:
receive patient training data for a plurality of patients;
create at least one decision model using the patient training data;
receive patient input data for at least one patient;
provide the patient input data as input to the at least one decision model;
obtain output from the decision model; and
generate at least one recommendation for patient treatment for presentation via a user interface based on the output of the decision model.

2. The computer system of claim 1, wherein the at least one decision model comprises a regression model.

3. The computer system of claim 1, wherein the at least one decision model is created using a set of one or more random decision forests.

4. The computer system of claim 3, wherein the at least one decision model comprises two or more decision models, and wherein each of the decision models is created using a different random decision forest.

5. The computer system of claim 3, wherein the at least one decision model comprises four decision models, wherein the set of one or more random decision forests comprises four random decision forests, and wherein the decision models are for obtaining decisions on, respectively, (1) continuing or canceling an ovarian stimulation cycle, (2) if the cycle is to continue, whether to trigger egg retrieval or to continue stimulation, (3) if stimulation is to be continued, the date of the next monitoring appointment, and (4) if stimulation is to be continued, whether the dose of stimulation medication is to be increased or decreased, or remain the same.

6. The computer system of claim 4, wherein data points for the decision forests include one or more of the following inputs: age of the patient when the cycle began; body mass index of the patient when the cycle began; protocol being followed for the cycle; patient diagnosis category; visit number in the cycle; number of days elapsed since the beginning of the cycle; measured estradiol levels; previously administered stimulation doses; measured diameters of one or more developing follicles.

7. The computer system of claim 2, wherein the output from the at least one decision model comprises one or more confidence percentages for one or more potential outcomes, and wherein the at least one recommendation is generated based on the one or more confidence percentages.

8. A computer-implemented method using a repository of patient medical records in supporting clinical decision-making during in vitro fertilization (IVF), comprising:
receiving patient data from a patient electronic medical record (EMR) database associated with treatment of infertility wherein the patient data comprises age, diagnosis, lab testing data prior to start of an IVF cycle, and observations during a process of ovarian stimulation;
identifying, in said patient electronic medical record database, information concerning different treatments previously employed for treating infertility; and
based on the identified information, generating an order for patient treatment, wherein the order is associated with protocol selection, dose of medication, date of follow up, or cycle stop.

9. The computer-implemented method of claim 8, wherein generating the order for patient treatment comprises providing the patient data as input to at least one decision model, wherein the at least one decision model is created using a set of one or more random decision forests.

10. The computer-implemented method of claim 8, wherein the information concerning different treatments includes one or more of diagnosis data, laboratory data, maternal age, sperm parameters, and prior different treatments.

11. The computer-implemented method of claim 8, wherein the information concerning different treatments includes resource consumption characteristics, and wherein said resource consumption characteristics are selected from a group consisting of: cost or duration of a treatment or course of treatment, physician or staff hours, cost or quantity of medicine, cost or duration of equipment usage, cost or duration of inpatient stay, or cost or duration of home care.

12. The computer-implemented method of claim 8, further comprising transmitting the order to a patient computing device or a clinician computing device, wherein the transmitting may be performed immediately or held pending approval by a physician.

13. The computer-implemented method of claim 8, wherein the order includes (a) an order for a medical test to be made for a patient, (b) an order for a pharmacological prescription for a patient, (c) an order for a service to be performed for a patient SER (sonographic egg retrieval), (d) an order for diagnostic imaging to be performed for a patient, (e) an order for surgical treatment for a patient, or (f) an order for a form of physiological therapy to be performed on a patient.

14. The computer-implemented method of claim 8 further comprising:
analyzing the different treatment information to determine a difference in diagnosis or treatment associated with said order and corresponding previous diagnoses and treatments recorded for other patients.

15. The computer-implemented method of claim 14 further comprising:
generating a report of said analysis for display in a user interface.

16. A computer-readable storage medium having stored thereon computer-executable instructions configured to cause one or more computing devices to perform the method of claim 8.

17. (canceled)

18. A computer system configured to use a repository of patient medical records in supporting clinical decision-making, the system comprising:
one or more computing devices programmed to, at least:
receive, from a source computing device, data representing an order associated with treatment of a medical condition;
interpret the order to determine search criteria for use in identifying records related to the medical condition;
initiate a search of a database of patient medical records based on the search criteria to identify treatment information concerning different treatments previously employed for treating the medical condition; and
transmit the treatment information to the source computing device.

19. The computer system of claim 18 further comprising the database of patient medical records:

20. The computer system of claim 18 wherein at least one of the one or more computing devices is programmed to provide a user interface for use in supporting clinical decision making, the user interface configured to receive user input and generate data representing the order associated with treatment of the medical condition.

21. The computer system of claim 18 wherein the order is generated by providing the patient data as input to at least one decision model created using a set of one or more random decision forests.
